(19) United States
(12) Patent Application Publication
(10) Pub. No.: US 2009/0148537 A1
(43) Pub. Date: Jun. 11, 2009

(54) MEDICAL COMPOSITION

(75) Inventor: Peter Charles Molan, Hamilton (NZ)

Correspondence Address: KNOBBE MARTENS OLSON & BEAR LLP, 2040 MAIN STREET, FOURTEENTH FLOOR, IRVINE, CA, 92614, US

(73) Assignee: Comvita Limited

(21) Appl. No.: 12/066,077

(22) PCT Filed: Sep. 5, 2006
(86) PCT No.: PCT/NZ2006/000234

§ 371 Date: Sep. 2, 2008

(30) Foreign Application Priority Data
Sep. 6, 2005 (NZ) 542258

(51) Int. Cl. A61K 35/64; A61K 31/70; A61K 31/047

(52) U.S. Cl. 424/537; 514/23; 514/724

(57) ABSTRACT

The present invention relates to a method of preparing a medical composition, including the steps of preparing a treatment composition, and adding at least one viscosity increasing agent to the treatment composition, characterized in that the viscosity increasing agents has a property of increasing or at least maintaining the viscosity of the treatment composition after it is applied to a wound.

### TECHNICAL FIELD

This invention relates to a medical composition.

In particular the invention relates to improvements in the application of honey based compositions to wounds.

### BACKGROUND ART

Honey has been widely used in the medical treatment of wounds for thousands of years. It possesses properties which are beneficial in the treatment of wounds such as antibacterial activity, anti-inflammatory activity, it stimulates the growth of cells which repair injured tissues, and it provides a moist healing environment optimal for wound healing.

Simple methods of applying honey to wounds are varied and range from soaking cotton gauze in honey or forming an ointment or “rubbery gel”.

Traditionally, the application of honey to wounds has been difficult because of the nature of a honey composition. At body temperatures a honey composition tends to soften. In combination with the wound exudate the composition becomes a very fluid substance and can run out of the wound, not staying in place, making these applications inefficient and messy.

Commercially products are available for treating wounds based on honey which try to overcome these problems. These include turning honey into a rubbery gel or an ointment, or applying a honey composition to a bandage.

An ointment is an effective way of preventing honey from running off a wound as it is raised to body temperature. However, ointments can be diluted and the honey washed away by body fluids present in a wound.

Honey impregnated fibre dressings are known. A weakness of these is that wound exudate dilutes and washes away the honey therein

Cognated New Zealand applications 501687 and 501748 relate to the preparation of a medical composition for dressing wounds through the combination of one or more honeys with a gelling agent. This turns the composition into a formable and/or pliable solid that can be readily moulded to fit a wound.

This invention has potentially some difficulty with the application of the honey composition to a wound. The composition must be shaped to the wound to which it is to be applied. This makes application difficult and inefficient. The honey composition is not capable of being applied to a wound cavity where there is a small opening.

Products are known (e.g. Medihoney™ Wound Gel) which are thickened by wax. However this product teaches away from the aims of the present invention as instead of absorbing exudates, the product is washed away from the wound by the exudates.

It would be advantageous to have a honey composition which allowed easy application to a variety of wounds. This new honey composition would be a stable composition at body temperature. If diluted by body fluids present in wounds it would not become prone to running or washing away from the wound. Intrasite™ hydrogel wound dressing is produced by Smith & Nephew. This product has a good reputation and is highly endorsed by healthcare professionals. Because of this, Intrasite™ hydrogel has been chosen as a viscosity bench mark for the present invention. However, this gel has no ability to absorb wound exudate without the gel decreasing in viscosity. The present invention addresses this problem that is common to all such hydrogel products.

It is thought that other treatment compositions could also have this problem as well.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a method of preparing a medical composition, including the steps of:
a) preparing a treatment composition, and
b) adding a viscosity increasing agent to the treatment composition, characterised in that the viscosity increasing agent has the property of increasing or at least maintaining the viscosity of the treatment composition after it is applied to a wound.

The term wound should be taken to mean any treatment area or a body, whether human or animal.

According to another aspect of the present invention there is provided a medical composition as prepared by the foregoing method.

According to further aspect of the present invention there is provided a wound treatment dressing which contains a medical composition as described above.

According to yet another aspect of the present invention there is provided a wound treatment applicator which contains a medical composition as described above.

According to yet another aspect of the present invention there is provided a method of treatment of wounds characterised by applying a medical composition as described above.

It is envisaged that in most embodiment of the present invention, it will be the body fluid (exudate) of the patient (which will be above ambient temperature) that causes the desired increase or maintenance in viscosity after application of the composition to the treatment area.

Preferred embodiments of the present invention has the treatment composition including primarily honey. However the composition may include instead of or in addition to the honey polyalcohols (e.g. glycerol, propylene glycol) and/or sugar syrup consisting of one of more sugars (e.g. glucose) and/or sugar alcohols (e.g. xylitol). Other wound compatible water binding compositions may be use which will prevent the viscosity increasing agent from hydrating until wound fluid is absorbed.

In preferred embodiments of the present invention the honey composition may consist of one or more honeys. It is envisaged by the inventor that at least one of these honeys will be Manuka honey.

However, this should not be seen as a limitation on the current invention. Other embodiments envisioned include a honey other than Manuka honey selected to have a high level of antibacterial activity, anti-inflammatory activity, antioxidant activity or other therapeutic activities.

In a preferred embodiment the honey composition may consist of, or include a UMF fraction.

In a preferred embodiment the UMF fraction may be extracted from honey derived from manuka (Leptospermum scoparium) or other Leptospermum species.

In alternative embodiments the bioactive compound or UMF fraction may be extracted from any part of the Leptospermum plant.

The viscosity agents may be any substance which does not substantially increase the viscosity of the honey composition until after it is applied to a wound. The presence of the viscosity increasing agent will increase the viscosity of the honey composition to a point where it does not “run” from a wound after application. This is necessary to ensure that the medical composition can adequately treat the wound.

It is envisaged that in preferred embodiments the viscosity agent will function only after water has been absorbed.

In some embodiments, the viscosity of the composition may only be maintained, as a consequence of the ‘viscosity increasing agent’ soaking up exudate at a rate that the composition's flow characteristics remain largely unchanged.

The challenge is to find an agent that would not hydrate at low water activity (characteristic of honey) but would hydrate as the water activity increases when body fluid exudes from a wound into the paste.

It is also envisaged by the inventor that the honey composition may include other additives, including but not limited to a calcium salt, an antifungal agent, or an inert powder to increase the firmness of the paste to prevent it running off a wound before it absorbs wound fluid and becomes a gel.

Further it should be appreciated that the starting viscosity of the composition is preferably that of a paste that can be readily applied—say squeezed from a tube.

The subsequent water binding of the viscosity increasing agent will then provide a desired viscosity profile for the paste.

The two variables within this have been the absorptive capacity of the paste and the pressure the paste can withstand before it is caused to flow off the wound. These are necessary components as the wound dressing should be able to absorb exudates from the wound, and the paste needs to able to withstand a suitable amount of pressure to be able to remain under the bandage without leaking.

The two variables have been measured with a group of different gelling agents in an attempt to select the most suitable gelling agent for the paste. The different gelling agents tried included Carboxymethyl Cellulose, Citrus Pectin, Guar Gum, Hydroxypropyl Methyl Cellulose, Metolose, Methyl Cellulose, Natrosol and Xanthan Gum. Carboxymethyl Cellulose and Xanthan Gum were found to solidify the honey after incubation for 18 hours at 37° C. Therefore no measurements were made with these, as this premature gelling is likely to give a short shelf-life for the paste. The ratio of gel powder to honey was an important aspect to this project as it determines the amount of exudate the paste can absorb. However, a balance needs to be maintained, the paste needs to be able to absorb a reasonable amount of exudate without losing the ability to withstand the pressure of the bandage, but must also be soft enough to be squeezed out of a tube for application.

In the preferred embodiments of the present invention the viscosity increasing agent is low substitution methylcellulose However, this should not be seen as a limitation on the present invention. Other embodiments for the viscosity increasing agent envisaged include Metaloseand Guar Gum. It is also envisaged that a judicious mixture of viscosity-increasing agents may be used to maintain the desired viscosity profile over a range of volumes of exudate absorbed.

In some embodiments a combination of viscosity increasing agents may be used.

In preferred embodiments of the present invention the viscosity increasing agent may be applied to the honey based composition as a powder. This addition of solid particles binds with free water in the exudate forming a paste which has decreased flow characteristics. However, this should not be seen as a limitation on the present invention.

The final viscosity of the composition after application will vary according to the amount of exudates produced and possibly the temperature of the patient. However, for typical usage it is envisaged that the viscosity will be in the order of (or greater than) that exhibited by Intrasite hydrogel—say in the order of withstanding a pressure of up to 2.0 g/cm² without being made to flow by the pressure applied.

In another embodiment of the present invention the medical composition may have other substances added to it to increase the solidity of the paste before it is applied to a wound. These substances are only applied to the composition to ensure that it does not run from a wound after application. These additives will not significantly alter the viscosity of the composition before it absorbs wound excaudate. The benefit of using these additives is that it will ensure that the honey composition does not run from a wound before it is able to absorb wound exudate but can still be easily applied to a variety of different wound sizes and openings.

Embodiments envisaged for these additives include an inert powder that is compatible with application to a wound surface. For example this may include starch or glucose.

In another embodiment of the present invention a non-aqueous fluid may be added to the composition to reduce the firmness of the composition prior to application to a wound. This may be necessary when a high ratio of the viscosity increasing agent is used to prevent the composition running from a wound.

A high ratio of the viscosity increasing agent compound may be needed when a wound is weeping or contains a lot of moisture.

Embodiments envisaged for the non-aqueous fluid include, but are not limited to glycerol or propylene glycol.

However, this should not be seen as a limitation on the present invention. Any non-aqueous fluid that can reduce the firmness of the paste prior to application to a wound in envisaged.

Another liquid water-binding composition could be used which will prevent the powdered gelling agent from hydrating until the wound fluid is absorbed.

With reference to the following description further aspects of the present invention will become apparent.

As a general procedure, finely powdered methylcellulose is blended with a honey composition.

The ratio of methylcellulose to honey composition depends on the end product required.

However, a ratio (by weight) of 1 part methylcellulose to 5 parts of honey gives a paste that can absorb approximately 4 times its own weight of wound fluid and still remain on a wound.

After application of the medical composition to a wound, absorption of moisture from the wound by the medical composition will act to increase viscosity of the medical composition. This ensures that the medical composition will not run from the wound once it is diluted by body fluid as often occurs when honey based products are used to treat wounds.

This medical composition has the advantage that it allows easy application to a variety of wounds. These wounds may be different sizes and/or have different types of openings. This will allow the medical composition to be widely used in a variety of situations.

These may include filling cavities from extracted teeth, “injection” into the gum margins to treat gingivitis and periodontal disease, intra-vaginal use for the treatment of vaginal infections and cervical ulcers, “injection” into the uterus for treatment of uterine infections, use as an enema for treatment of ulcerative colitis with honey, or as a teat seal for the prevention of mastitis in dairy cows. However, this should not be seen as a limitation on the use of the current invention.

Other embodiments envisioned are where a honey based medical composition is desired and it would be advantageous that its viscosity is altered after application to a wound or cavity.

### BEST MODES FOR CARRYING OUT THE INVENTION

Tests undertaken to determine the best composition for the present invention are given below.

Eight powdered gelling agents, namely Carboxymethyl Cellulose, Citrus Pectin, Guar Gum, Hydroxypropyl Methyl Cellulose (viscosity 3,500-5,600 cP), Metolose (60SH-4000), Methyl Cellulose (low substitution), Natrosol and Xanthan Gum, were tested at a ratio of 0.2 g of gelling powder to every 1.0 g of honey. Some further tests were also carried out on three of these (Guar Gum, Metolose, and Hydroxypropyl Methyl Cellulose) at a ratio of 0.15 g of gelling powder to every 1.0 g of honey and at a ratio of 0.4 g of gelling powder to every 1.0 g of honey.

### 1. Methodologies

1. Glass Plate Method for determining viscosity of gel: This is a measurement of the pressure a paste/gel can withstand before being squeezed away.
   Gel powders were passed through 106 μm sieve.
   Appropriate ratio of gel powder and honey were mixed together (i.e. 0.2 g gel: 1.0 g Honey)
   Portions of the paste thus formed were mixed with appropriate volumes of water to get the required ratios of water to paste (i.e. 1.0 ml/g, 2.0 ml/g, 3.0 ml/g, 4.0 ml/g)
   The mixtures thus prepared were incubated for 18 hours at 37° C. in closed containers.
   Following this incubation, 5.0 g of each paste or the gel formed from the mixture of paste and water was placed onto the centre of a glass plate and a second plate of known weight was placed on top. This was carried out in triplicate for each paste or gel.
   The sandwiches of paste/gel between plates thus prepared were incubated for 18 hours at 37° C. on a level surface.
   After they had been incubated, the sandwiches of paste/gel between plates were gently transferred to a photocopier to obtain an image giving an outline of the extent of the spread of the gel.
   Using image-analysis software, the outline was traced, filled in, and the area of the gel was measured.
   From the area of spread of the gel and the weight of the glass plate that had been laid on top of the gel could be calculated the maximum pressure which the gel could withstand.

$$Pressure = \frac{Force}{Area}$$

### 2. Results

Honey mixed with Carboxymethyl Cellulose, and Xanthan Gum, without any water added, became solid after incubation at 37° C. Natrusol and Citrus Pectin became very viscous without any water added after incubation at 37° C. This indicated that the pastes made with these five gelling agents were likely to have a short shelf-life before becoming too viscous to squeeze out of a tube. Thus these gelling agents were deemed to be unsuitable for the invention.

Metolose, Hydroxypropyl Methyl Cellulose and Guar Gum were found to be suitable. The measurements obtained with the gelling agents tested are shown in Table 1.

TABLE 1
Maximum pressure (g/cm²) withstood with 0.2 g gelling agent added to 1 g honey

| | | | | | | |
|---|---|---|---|---|---|---|
| Gelling agent | After 18 | After 18 | After 18 | After 18 | After 18 | After 18 |
| | hours at | hours at | hours at | hours at | hours at | hours at |
| | 37° C. | 37° C., | 37° C., | 37° C., | 37° C., | 37° C., |
| | with no | water | water | water | water | water |
| | water | added, 1 | added, 2 | added, 3 | added, 4 | added, 6 |
| | added | ml: 1 g | ml: 1 g | ml: 1 g | ml: 1 g | ml: 1 g |
| Carageenan | Solid | | | | | |
| Carboxymethyl | Solid | | | | | |
| cellulose | | | | | | |
| Citrus pectin | 47.85 | 2.53 | Watery | | Watery | Watery |
| Guar gum | 10.33 | | 7.21 | | 3.4 | 2.18 |
| Hydroxypropyl | | | 2.77 | 2.23 | 2.14 | |
| methyl cellulose | | | | | | |
| Karaya gum | Solid | | | | | |
| Metolose | 6.86 | 11.37 | | | | |
| Methyl cellulose | 3.15 | 8.09 | 3.36 | 3.41 | 3.39 | |
| Natrosol | 40.54 | 9.76 | 2.42 | | | |
| Xanthan gum | Solid | | | | | |

Maximum pressure (g/cm²) withstood with 0.15 g gelling agent added to 1 g honey

| | | | | | |
|---|---|---|---|---|---|
| Gelling agent | After 18 | After 18 | After 18 | After 18 | After 18 |
| | hours at | hours at | hours at | hours at | hours at |
| | 20° C. | 37° C. | 37° C., | 37° C., | 37° C., |
| | with no | with no | water | water | water |
| | water | water | added, 1 | added, 2 | added, 3 |
| | added | added | ml: 1 g | ml: 1 g | ml: 1 g |
| Guar gum | 6.85 | 15.21 | 8.77 | 4.78 | 2.65 |
| Hydroxypropyl | 5.09 | 3.21 | | 2.09 | |
| methyl cellulose | | | | | |
| Metolose | 4.00 | 3.26 | | 2.82 | 2.01 |

Maximum pressure (g/cm²) withstood with 0.4 g gelling agent added to 1 g honey

| | | | | | |
|---|---|---|---|---|---|
| Gelling agent | After 18 | After 18 | After 18 | After 18 | After 18 |
| | hours at | hours at | hours at | hours at | hours at |
| | 37° C. | 37° C., | 37° C., | 37° C., | 37° C., |
| | with no | water | water | water | water |
| | water | added, 1 | added, 2 | added, 3 | added, 4 |
| | added | ml: 1 g | ml: 1 g | ml: 1 g | ml: 1 g |
| Guar gum | 37.76 | 32.73 | 16.19 | 16.35 | 6.21 |
| Hydroxypropyl | 18.95 | 11.71 | 11.21 | 3.68 | 1.93 |
| methyl cellulose | | | | | |
| Metolose | 13.98 | | 15.99 | 7.30 | 3.44 |

It should be noted that the measurements were made with a fairly high ratio of gelling agent to honey which results in pastes and gels that are of fairly high viscosity to withstand the pressure of compression bandaging. For other applications, where low viscosity hydro-gels are used (such as Intrasite™) a lower ratio of gelling agent to honey could suffice.

Aspects of the present invention have been described by way of example only and it should be appreciated that modifications and additions may be made thereto without departing from the scope of the appended claims.

1. A method of preparing a medical composition, including the steps of:
a. preparing a treatment composition, and
b. adding at least one viscosity increasing agent to the treatment composition, wherein the viscosity increasing agent is of the type and quantity that has the property of increasing the viscosity of the treatment composition only after it is applied to a wound.

2. A method of preparing a medical composition as claimed in claim 1 wherein the treatment composition includes honey.

3. A method of preparing a medical composition as claimed in claim 2 wherein the treatment composition includes Manuka honey.

4. A method of preparing a medical composition as claimed in claim 1 wherein the treatment composition includes sugar syrup.

5. A method of preparing a medical composition as claimed in claim 1 wherein the treatment composition includes glycerol.

6. A method of preparing a medical composition as claimed in claim 1 which includes a calcium salt.

7. A method of preparing a medical composition as claimed in claim 1 wherein the viscosity increasing agent includes Methylcellulose.

8. A method of preparing a medical composition as claimed in claim 1 wherein the viscosity increasing agent includes Guar Gum.

9. A method of preparing a medical composition as claimed in claim 1 wherein the viscosity increasing agent includes Hydroxypropyl Methyl Cellulose.

10. A method of preparing a medical composition as claimed in claim 1 wherein the viscosity increasing agent includes hydroxyethylcellulose.

11. A method of preparing a medical composition as claimed in claim 1 wherein the viscosity increasing agent includes at least one high substitution methylcellulose.

12. A method of preparing a medical composition as claimed in claim 1 wherein the viscosity increasing agent is in powder form.

13. A method of preparing a medical composition as claimed in claim 1 which includes an additive that increases the firmness of the composition before it is applied to the wound.

14. A method of preparing a medical composition as claimed in claim 1 wherein the additive is starch.

15. A method of preparing a medical composition as claimed in claim 1 which includes a non aqueous fluid that decreases the firmness of the composition before it is applied to the wound.

16. A method of preparing a medical composition as claimed in claim 15 wherein the non aqueous fluid is glycerol.

17. A method of preparing a medical composition as claimed in claim 16 wherein the non aqueous fluid is propyleneglycol.

18. A medical composition as prepared by the method as claimed in claim 1.

19. A wound treatment dressing which contains a medical composition as claimed in claim 18.

20. A method of treating a wound characterized by the step of applying a medical composition as claimed in claim 18.

21-22. (canceled)
